(19) United States
(12) Patent Application Publication
(10) Pub. No.: US 2024/0141371 A1
(43) Pub. Date: May 2, 2024

(54) A PLANT WITH INCREASED DEFENSE RESPONSE, REDUCED DISEASE LEVELS AND METHODS TO GENERATE SAME

(71) Applicant: The State of Israel, Ministry of Agriculture & Rural Development, Agricultural Research Organization, Rishon Lezion (IL)

(72) Inventors: Maya BAR, Ramat Gan (IL); Meirav LEIBMAN, Tel Aviv (IL)

(21) Appl. No.: 18/280,587

(22) PCT Filed: Mar. 8, 2022
(86) PCT No.: PCT/IL2022/050256

§ 371 Date: Sep. 6, 2023

Related U.S. Application Data
(60) Provisional application No. 63/157,836, filed on Mar. 8, 2021.

(51) Int. Cl. C12N 15/82; C07K 14/415

(52) U.S. Cl. CPC C12N 15/8282; C07K 14/415

(57) ABSTRACT

This invention is related to a mutated plant line that harbors an impaired LeEIX1 (Lycopersicon esculentum ethylene inducing xylanase receptor 1) gene. The said mutated plant has an increased defense response and a reduction in disease levels upon pathogen exposure compared to a non-mutated plant of the same population. This invention is also related to the method for increasing a plant defense response and for reducing plant diseases levels, comprised by (a) designing at least one nucleic acid sequence configured to targeting and impairing a plant's LeEIX1 gene, (b) applying a gene editing process utilizing said at least one nucleic acid sequence, (c) obtaining at least one mutant plant line, harboring an impaired LeEIX1 gene, (d) growing said plant, and (e) inoculating said plant with at least one bacterial biological control agent.

### FIELD OF INVENTION

The present invention relates to the field of agriculture. More particularly, the present invention concerns increasing plant resistance and decreasing damage from pathogens' infection by means of gene editing via crispr/cas system.

### BACKGROUND OF INVENTION

Plant pathogens are the foremost yield limiting factor for many crops in open field and greenhouse cultivation systems (see Vitti A, Sofo A, Scopa A, Nuzzaci M. 2015), and this trend is expected to increase. Fungal and bacterial pathogens are the cause of significant tomato crop losses worldwide, generating devastating diseases. Pesticidal strategies can lack effectivity and are often a source of pollution and detrimental effects to consumer health, with many pesticides becoming increasingly banned worldwide. Induced resistance is recognized as an important mode of action to achieve biocontrol in vegetative tissues (see Kuc, 1987; Sequeira, 1983). Induced systemic resistance (ISR) caused by various micro-organisms can protect plants against soil or foliar pathogens (see Paulitz and Matta, 2000). In agriculture, the most environmentally friendly way to combat plant diseases is to make use of the innate immune system of plants, for instance by crossing into crop varieties polymorphic resistance genes that occur in natural populations of the crop plant or its close relatives. Plant pathogens, however, have co-evolved with their host plants and have developed ways to overcome the immune system. (see Houterman PM et al., 2008 Suppression of Plant Resistance Gene-Based Immunity by a Fungal Effector. PLoS Pathog 4(5): e1000061).

In view of this knowledge, developing novel methods for increasing crops' immunity by lifting immune suppression is a long-felt need.

The Trichoderma fungal protein elicitor EIX (ethylene induced xylanase), induces ethylene biosynthesis, electrolyte leakage, expression of PR proteins and the hypersensitive response (HR) in specific plant species and/or varieties (see Bailey et al., 1990, 1992; Ron et al., 2000; Elbaz et al., 2002). EIX was shown to specifically bind to the plasma membrane of responsive cultivars of both tomato and tobacco (see Hanania and Avni, 1997). The response to EIX in tobacco and tomato cultivars is controlled by a single dominant locus, termed LeEix (see Ron and Avni, 2004). The LeEix locus contains two receptors, LeEix1 and LeEix2, both belonging to a class of leucine-rich repeat cell-surface glycoproteins. Both receptors are able to bind the EIX elicitor while only the LeEix2 receptor mediates plant defense responses (see Kuc J. 1987). LeEix1 acts as a decoy receptor and attenuates EIX induced signaling of the LeEix2 receptor (see Sequeira L. 1983, Paulitz, T.C., Matta, A. 2000).

Considering that EIX acts to increase plant immunity through binding and downstream signaling of LeEIX2 (see Kuc J. 1987), and that LeEIX1 can act to block this immunity promoting downstream signaling (see Sequeira L. 1983, Paulitz, T.C., Matta, A. 2000), the inventors of the present invention hypothesized that removing/mutating LeEIX1 might result in stronger immune activation and enhancement of disease resistance.

In view of the prior art, and given the various challenges described above, there is still an unmet need for developing novel methods for enhancing plant defense responses and pathogen resistance.

### SUMMARY OF THE INVENTION

It is thus an object of the present invention to disclose a mutated plant line characterized by an increased defense response compared to a non-mutated plant of the same population, wherein said mutated plant harbors an impaired LeEIX1 (Lycopersicon esculentum ethylene inducing xylanase receptor 1) gene.

It is another object of the present invention to disclose a mutated plant line characterized by a reduction in disease levels compared to a non-mutated plant of the same population upon pathogen exposure, wherein said mutated plant harbors an impaired LeEIX1 (Lycopersicon esculentum ethylene inducing xylanase receptor 1) gene.

It is another object of the present invention to disclose the aforementioned mutated plant line, wherein said mutated plant line is homozygous to the impaired LeEIX1 gene.

It is another object of the present invention to disclose the aforementioned mutated plant line, wherein said mutated plant line expresses a non-functional, truncated LeEIX1 protein. It is another object of the present invention to disclose the aforementioned mutated plant line, wherein said plant is a member of the Solanaceae family.

It is another object of the present invention to disclose the aforementioned mutated plant line, wherein said plant is selected from a group consisting of Solanum lycopersicum, Nicotiana tabacum, Solanum tuberosum, Solanum melongena, Capsicum annum, Petunia hybrida, a LeEIX1 gene expressing plant, a plant expressing a LeEIX1 functional ortholog, and any combination thereof.

It is another object of the present invention to disclose the aforementioned mutated plant line, wherein said increased defense response is further increased upon interaction with either a pathogen or a bio control agent (BCA).

It is another object of the present invention to disclose the aforementioned mutated plant line, wherein the reduction in disease levels is further increased by a bio control agent (BCA).

It is another object of the present invention to disclose the aforementioned pathogen, wherein said pathogen is selected from a group consisting, viruses, bacteria, fungi, oomycetes, pests and any combination thereof.

It is another object of the present invention to disclose the aforementioned pathogen, wherein said pathogen is selected from a group consisting of: Botrytis, Sclerotinia, Sclerotium rolfsii, Alternaria, Pythium, Phytopthora, Fusarium, Oidium, Lasodiplodia, Penicillium, Aspergillus, Talaromyces, Macrophomina, Verticillium, Cladosporium, Clavibacter, Pseudomonas, Xanthomonas, Xyllela, Erwinia, Tobamoviruses, Tomato yellow leaf curl virus, Gemini viruses, Flies, Mites, Leafhoppers, Leaftniners.

It is another object of the present invention to disclose the aforementioned bio control agent, wherein said agent is selected from a group consisting of
a. insects,
b. microorganisms such as fungi, bacteria, oomycets, viruses,
c. chemicals such as nucleic acids, hormones, hormone analogs, natural products, organic compounds.

It is another object of the present invention to disclose the aforementioned bio control agent, wherein said agent is Trichoderma harzianum.

It is another object of the present invention to disclose the aforementioned bio control agent, wherein said agent is T39.

It is another object of the present invention to disclose the aforementioned mutated plant line, wherein said mutated plant line is mutated by means of a gene editing system.

It is another object of the present invention to disclose the aforementioned mutated plant line, wherein said gene editing system is selected from a group consisting of: gene editing agent, meganucleases, zinc finger nucleases, TALEN, Crispr/Cas and any combination thereof.

It is another object of the present invention to disclose the aforementioned mutated plant line, wherein said gene editing system is crispr/cas 9 system.

It is another object of the present invention to disclose a method for increasing a plant defense response, comprising the following steps:
a. designing at least one nucleic acid sequence configured to targeting and impairing the LeEIX1 gene,
b. applying a gene editing process utilizing said at least one nucleic acid sequence; and
c. obtaining at least one mutant plant line, harboring an impaired LeEIX1 gene.

It is another object of the present invention to disclose a method for increasing a plant defense response, comprising steps:
a. designing at least one nucleic acid sequence configured to targeting and impairing a plant's LeEIX1 gene,
b. applying a gene editing process utilizing said at least one nucleic acid sequence,
c. obtaining at least one mutant plant line, harboring an impaired LeEIX1 gene,
d. growing said plant,
e. inoculating said plant with at least one bacterial biological control agent.

It is another object of the present invention to disclose the aforementioned method, wherein said nucleic acid sequence is selected from a group consisting of DNA, RNA, tRNA, gRNA, cDNA, hybrid nucleic acid, or any combination thereof.

It is another object of the present invention to disclose the aforementioned method, wherein said nucleic acid sequence is gRNA of the CRISPR/Cas9 gene editing system.

It is another object of the present invention to disclose the aforementioned method, wherein said plant is a member of the Solanaceae family.

It is another object of the present invention to disclose the aforementioned method, wherein said plant is selected from a group consisting of Solanum lycopersicum, Nicotiana tabacum, Solanum tuberosum, Solanum melongena, Capsicum annum, Petunia hybrida, a LeEIX1 expressing plant, a plant expressing a LeEIX1 ortholog, and any combination thereof.

It is another object of the present invention to disclose the aforementioned method, wherein said mutant plant line is homozygous to the impaired LeEIX1 gene.

It is another object of the present invention to disclose the aforementioned method, wherein said mutant plant line expresses a non-functional, truncated LeEIX1 protein.

It is another object of the present invention to disclose the aforementioned method, wherein said mutant plant line possesses at least one increased defense response.

It is another object of the present invention to disclose the aforementioned method, wherein said defense response is further increased upon interaction with either a pathogen or a bio control agent (BCA).

It is another object of the present invention to disclose the aforementioned method, wherein said pathogen is selected from a group consisting of viruses, bacteria, fungi, oomycetes, pests and any combination thereof.

It is another object of the present invention to disclose the aforementioned method, wherein said pathogen is selected from a group consisting of: Botrytis, Sclerotinia, Sclerotium rolfsii, Alternaria, Pythium, Phytopthora, Fusarium, Oidium, Lasodiplodia, Penicillium, Aspergillus, Talaromyces, Macrophomina, Verticillium, Cladosporium, Clavibacter, Pseudomonas, Xanthomonas, Xyllela, Erwinia, Tobamoviruses, Tomato yellow leaf curl virus, Gemini viruses, Flies, Mites, Leafhoppers, Leaftniners and any combination thereof.

It is another object of the present invention to disclose the aforementioned method, wherein said at least one bio control agent is selected from a group consisting of
a. insects,
b. microorganisms such as fungi, bacteria, oomycets, viruses,
c. chemicals such as nucleic acids, hormones, hormone analogs, natural products, organic compounds.

It is another object of the present invention to disclose the aforementioned fungi, wherein said fungi is Trichoderma Spp.

It is another object of the present invention to disclose the aforementioned bacteria, wherein said bacteria is selected from a group consisting of Bacillus Spp., Pseudomonas Spp.

It is another object of the present invention to disclose the aforementioned method, wherein said at least one biological control agent is Trichoderma harzianum.

It is another object of the present invention to disclose the aforementioned method, wherein said at least one biological control agent is T39.

It is another object of the present invention to disclose the aforementioned method, wherein said bio control agent is selected from a group consisting of from a group consisting of
a. insects,
b. microorganisms such as fungi, bacteria, oomycets, viruses,
c. chemicals such as nucleic acids, hormones, hormone analogs, natural products, organic compounds

It is another object of the present invention to disclose the aforementioned fungi, wherein said fungi is Trichoderma Spp.

It is another object of the present invention to disclose the aforementioned bacteria, wherein said bacteria is selected from a group consisting of Bacillus Spp., Pseudomonas Spp.

It is another object of the present invention to disclose the aforementioned method, wherein said bio control agent is Trichoderma harzianum.

It is another object of the present invention to disclose the aforementioned method, wherein said bio control agent is T39.

It is another object of the present invention to disclose a method for reducing plant diseases levels, comprising the following steps:
a. designing at least one nucleic acid sequence configured to targeting and impairing the LeEIX1 gene,
b. applying a gene editing process utilizing said at least one nucleic acid sequence; and
c. obtaining at least one mutant plant line, harboring an impaired LeEIX1 gene.

It is another object of the present invention to disclose a method for reducing plant diseases levels, comprising steps:
a. designing at least one nucleic acid sequence configured to targeting and impairing a plant's LeEIX1 gene,
b. applying a gene editing process utilizing said at least one nucleic acid sequence,
c. obtaining at least one mutant plant line, harboring an impaired LeEIX1 gene,
d. growing said plant,
e. inoculating said plant with at least one bacterial biological control agent.

It is another object of the present invention to disclose the aforementioned method, wherein said nucleic acid sequence is selected from a group consisting of DNA, RNA, tRNA, gRNA, cDNA, hybrid nucleic acid, or any combination thereof.

It is another object of the present invention to disclose the aforementioned method, wherein said nucleic acid sequence is gRNA of the CRISPR/Cas9 gene editing system.

It is another object of the present invention to disclose the aforementioned method, wherein said plant is a member of the Solanaceae family.

It is another object of the present invention to disclose the aforementioned method, wherein said plant is selected from a group consisting of Solanum lycopersicum, Nicotiana tabacum, Solanum tuberosum, Solanum melongena, Capsicum annum, Petunia hybrida, a LeEIX1 expressing plant, a plant expressing a LeEIX1 ortholog, and any combination thereof.

It is another object of the present invention to disclose the aforementioned method, wherein said mutant plant line is homozygous to the impaired LeEIX1 gene.

It is another object of the present invention to disclose the aforementioned method, wherein said mutant plant line expresses a non-functional, truncated LeEIX1 protein.

It is another object of the present invention to disclose the aforementioned method, wherein said mutant plant line possesses reduction in disease levels upon exposure to at least one pathogen.

It is another object of the present invention to disclose the aforementioned method, wherein said reduction in disease levels is further increased upon interaction with a bio control agent (BCA).

It is another object of the present invention to disclose the aforementioned method, wherein said at least one bio control agent is selected from a group consisting of
a. insects,
b. microorganisms such as fungi, bacteria, oomycets, viruses,
c. chemicals such as nucleic acids, hormones, hormone analogs, natural products, organic compounds.

It is another object of the present invention to disclose the aforementioned fungi, wherein said fungi is Trichoderma Spp.

It is another object of the present invention to disclose the aforementioned bacteria, wherein said bacteria is selected from a group consisting of Bacillus Spp., Pseudomonas Spp.

It is another object of the present invention to disclose the aforementioned method, wherein said at least one biological control agent is Trichoderma harzianum.

It is another object of the present invention to disclose the aforementioned method, wherein said at least one biological control agent is T39.

It is another object of the present invention to disclose the aforementioned method, wherein said bio control agent is selected from a group consisting of from a group consisting of
a. insects,
b. microorganisms such as fungi, bacteria, oomycets, viruses,
c. chemicals such as nucleic acids, hormones, hormone analogs, natural products, organic compounds

It is another object of the present invention to disclose the aforementioned fungi, wherein said fungi is Trichoderma Spp.

It is another object of the present invention to disclose the aforementioned bacteria, wherein said bacteria is selected from a group consisting of Bacillus Spp., Pseudomonas Spp.

It is another object of the present invention to disclose the aforementioned method, wherein said bio control agent is Trichoderma harzianum.

It is another object of the present invention to disclose the aforementioned method, wherein said bio control agent is T39.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention. These and other features, aspects, and advantages of the present invention are better understood when the following Detailed Description is read with reference to the accompanying drawings, wherein:

FIG. 1a depicting a schematic presentation of the DNA and amino acid sequences of the present invention.

FIG. 1b depicting a schematic presentation of the protein structure and domains of the present invention.

FIG. 1c depicting a schematic presentation of the protein sequences of the present invention.

FIG. 2a depicting a graphical presentation of a Harvest Index (HI) of the present invention.

FIG. 2b depicting a graphical presentation of the total soluble sugar of the present invention.

FIG. 2c depicting a graphical presentation of the fruit weight per plant of the present invention.

FIG. 2d depicting a graphical presentation of the average total number of fruits per plant of the present invention.

FIG. 2e depicting a graphical presentation of an analysis of growth and development parameters of the present invention.

FIG. 3a depicting a graphical presentation of a Harvest Index (HI) of the present invention.

FIG. 3b depicting a graphical presentation of the total soluble sugar of the present invention.

FIG. 3c depicting a graphical presentation of the fruit weight per plant of the present invention.

FIG. 3d depicting a graphical presentation of the average total number of fruits per plant of the present invention.

FIG. 4, FIG. 5a-FIG. 5e depicting schematic and graphical presentations of a comparison of disease percentage and lesion area and pathogen levels of the present invention.

FIG. 6a depicting a graphical presentation of the ethylene induction after wounding and EIX elicitation of the present invention.

FIG. 6b depicting a graphical presentation of the conductivity following wounding or EIX treatment of the present invention.

FIG. 6c and FIG. 6d depicting a graphical presentation of the ROS production immediately after EIX application.

FIG. 6e depicting a graphical presentation of gene expression analysis of the defense genes PI2, Pti-5, PR1a and beta-glucanase of the present invention.

FIG. 7a and FIG. 7b depicting a graphical presentations of the ROS (reactive oxygen species) of the present invention.

FIG. 8 depicting a graphical presentation of the gene expression analysis of PRR genes of the present invention.

FIG. 9a-f depicting a graphical presentations of the gene expression analysis of defense genes in M82 and leeix1 plants of the present invention.

FIG. 10 depicting schematic and graphical presentation of the lesion area of the present invention.

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide A method for increasing plant pathogen induced defense response to pathogenic microorganisms and a genome-edited plant characterized by exhibiting induced defense response to pathogenic microorganisms.

The term “gene editing” refers hereinafter to a type of genetic engineering in which DNA undergoes modifications, insertions, deletions or replacement in a genome.

The term “CRISPER/Cas9” refers hereinafter to a gene editing technique. Said technique enables gene editing in vivo with extremely high precision. It is based on a synthetic guide RNA (gRNA) delivering the Cas9 nuclease to a specific desired location in the cell's genome, allowing deletions or insertion of specific nucleotides into existing genes in vivo.

The term “gRNA/guide RNA” refers hereinafter to a synthetic RNA sequence, which is an integral component of the gene editing CRISPR/Cas9 system. It is a non-coding short RNA sequences that first binds to the Cas9 endonuclease and guides it to a specific location in the cell's genome, then binds to a complementary target DNA sequences where the Cas9 cleaves the target DNA strand.

The term “homozygous line” refers hereinafter to a homozygous plant which maintains a high degree of consistency for particular characters determined by the gene throughout the subsequent generations, true to type progenies-pure lines (see Nishat et al. J Biol Methods. 2016; 3(3)).

The term “knockout” refers hereinafter to a gene that is made inoperative (knockout) by means of gene editing or genetic engineering, or to the organism that carries said inopertaive gene. The term “protospacer adjacent motif (PAM) site” refers hereinafter to a short DNA sequence that follows the DNA region targeted for cleavage by the CRISPR-Cas9. The PAM is required for a Cas nuclease to cut, and is generally found 3-4 nucleotides downstream from the cut site.

The term “plant” refers hereinafter to any part of a plant (roots, stem, foliage, fruits, bulbs) that can be cultivated and harvested extensively to be used as a commodity or for sustainability.

The term “defense response” or “plant defense response” refers hereinafter to an activity of the plant's immune system, which can be triggered by microbial molecules. In some cases, plant pattern recognition receptors (PRRs) sense or detect different types of microbial molecules, generating a response by activation of a defense mechanism. Typical manifestations of plant defense include, but are not limited to: Ethylene biosynthesis, defense gene expression, ion leakage, callose deposition, hypersensitive response (HR), PR protein activity, protease inhibitor activity.

The term “suppressor gene” refers hereinafter to a plant's immune system related gene that upon interaction with its ligand, namely a specific pathogen molecule, it downregulates or blocks the plant's pathogen induced defense response.

The term “population” refers hereinafter to a group of organisms of a species that interbreed and live in same place at the same time. They are capable of interbreeding or reproducing. The term “wild type (WT)” refers hereinafter to an organism, a phenotype, a genotype, or a gene that predominates in a natural population of organisms or strain of organisms in contrast to that of natural, laboratory, bioengineered mutant forms.

The term “increased defense response”, “increasing defense response” refers hereinafter to an increase of about 10% of at least one defense response selected from the group consisting of: ethylene production, ion leakage, expression of at least one defense gene, HR, callose deposition, all calculated as further described, all in comparison to a wild type (WT) plant. Increased defense response can lead to a decrease of about 10% of relative disease levels or absolute lesion/necrosis/diseased area, calculated as further described.

The term “nonsense amino acid”, “nonsense” refers hereinafter to the substitution of an amino acid in a protein sequence. The substitution of this amino acid results in the production of a different, and likely disfunction, protein.

The term “BCAs”, “Bio Control Agents” refers hereinafter to a plant-associated beneficial insects, chemicals and microorganisms. The BCAs mitigate the growth of phytopathogens by various modalities and can promote crop production and resistance to various plant diseases. Thus, provide an environment friendly substitute to chemical/synthetic herbicides and pesticides. BCAs induce disease resistance against challenges by a broad range of pathogens and parasites, including fungi, viruses, bacteria and insects in plants.

The term “T39”, refers hereinafter to the Trichoderma harzianum T39 isolate, a frequently used BCA, which promotes the induced systemic resistance (ISR)in various plant species. There are commercial formulations Trichoderma harzianum T39 available in the market.

The term “ortholog”, “functional ortholog” refers hereinafter to genes in different species that have evolved through speciation events. Generally, orthologs have the same biological functions in different species.

In a preferred embodiment of the present invention, the plant which genome was edited via the CRISPR/CAS9 system is tomato, however this disclosure concerns any plant that expresses the LeEIX1 gene, LeEIX1's homologue, or LeEIX1's orthologue.

In yet another preferred embodiment of the present invention, the plant whose genome was edited via the CRISPR/CAS9 system is tomato, but this disclosure concerns any plant that expresses an immune suppressor gene, which attenuates the immune response mechanism.

In yet another preferred embodiment of the present invention, mutating the EIX (ethylene induced xylanase) genes is carried out by the CRISPR/CAS9 genome-editing system. Nevertheless, this is merely a non-binding example, and any other method known in the art of molecular biology and genetic engineering can be employed. This includes for example gene editing agent, meganucleases, zinc finger nucleases, TALEN and any combination thereof.

### Example 1

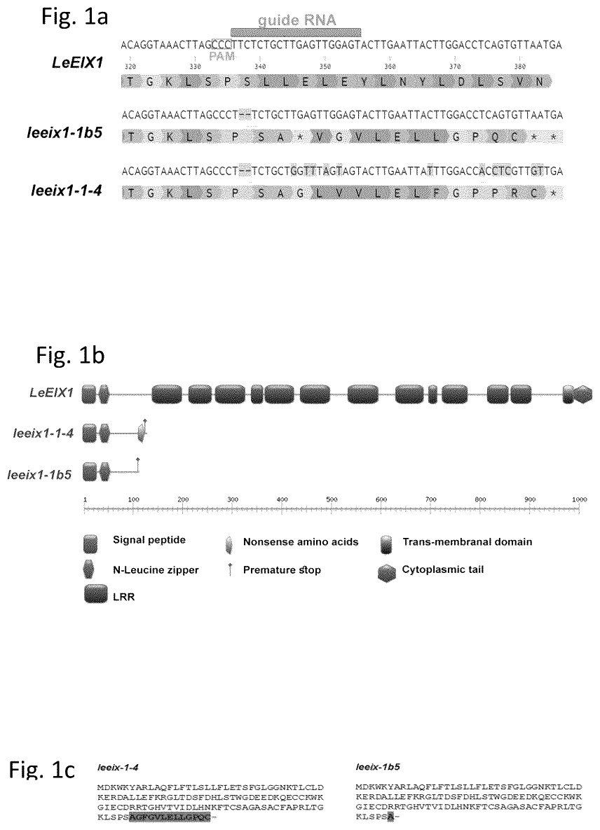

In order to produce the mutated homozygous tomato plant line of the present invention, the following steps were taken:
a. specifics gRNAs were designed to target only the LeEIX1 gene and not the LeEIX2 gene/the LeEIX genes derive from a single locus, both express leucine-rich repeat cell-surface glycoproteins and are able to bind the EIX elicitor. However, only the LeEix2 receptor mediates plant defense responses, while the LeEix1 acts as a decoy receptor and attenuates EIX induced signaling and defense response of the LeEix2 receptor. Reference is now made to FIG. 1a depicting gRNA/PAM site and resultant mutations in the LeEIX1 sequence.
b. Two independent homozygous lines LeEIX1 knockouts were obtained using the CRISPR/CAS9 system, resulting in a frame shift causing a premature stop codon:
   i. Leeix1-1b5 mutant with a two-base deletion (1-b5).
   ii. Leeix1-1-4 mutant with a two base deletion and additional insertions (1-1-4), at a PAM site −330 nucleotides after the LeEIX1 ATG.
c. The truncated proteins formed in the mutants contain only the signal peptide and N-terminal Leucine zipper, and do not have the LRR (Leucine-rich repeat) domains which are important for ligand recognition and protein-protein interactions, or the transmembranal domain required for PM (Plasma membrane) insertion. Reference is now made to FIG. 1b depicting Protein domain analysis of LeEIX1 alongside the mutant truncated proteins.
   i. Leeix1-1b5 mutant protein comprises of a truncated 113 amino acid protein from the N-terminus of the 1031 amino acid full LeEIX1.
   ii. leeix1-1-4 additionally having 13 “nonsesns” amino acids prior to the premature stop codon.

Thus, these truncated proteins formed would likely not be inserted in the membrane, or be able to bind the xylanase ligand or protein interactors-leading to null of LeEIX1 functionality. Reference is now made to FIG. 1c depicting predicted protein sequence of the mutants.

### Example 2

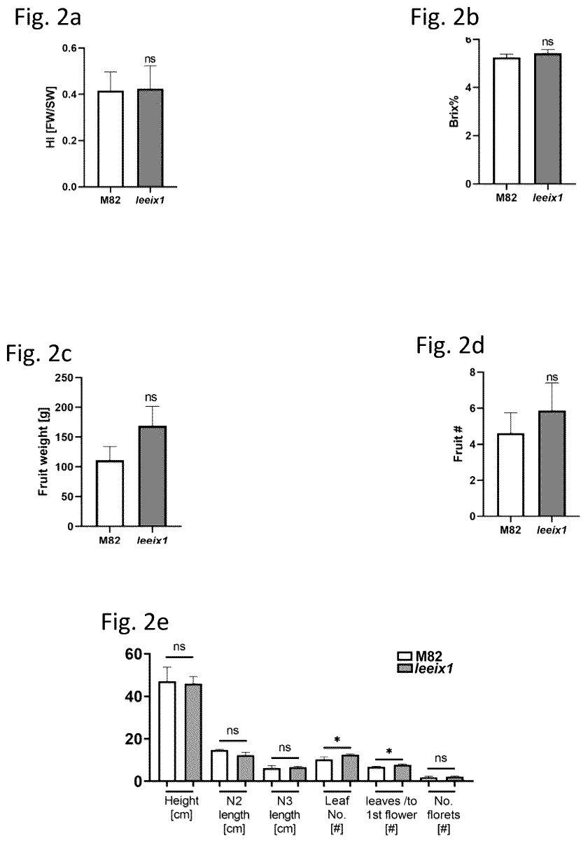

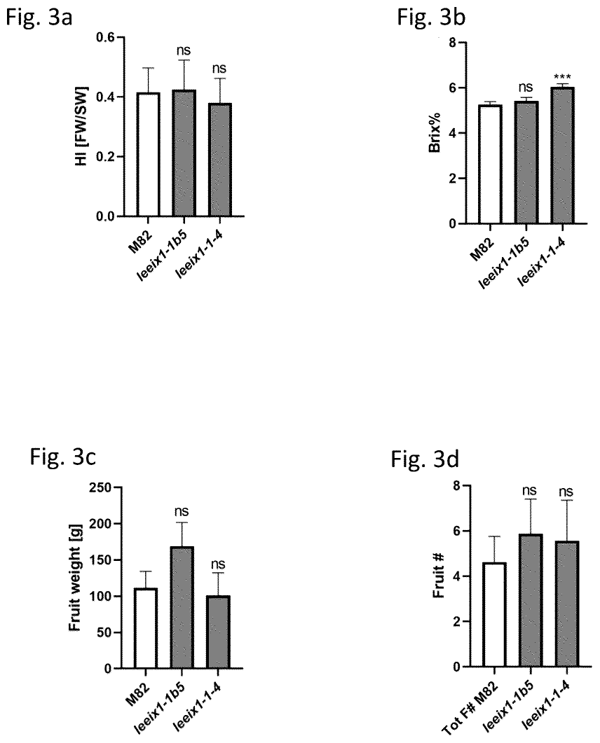

The present invention discloses a leEIX1 mutated homozygous plant line exhibiting similar agricultural traits as their wild type M82 background line, for example, developmental progression, agricultural quality, yield, and tomato quality Comparing agricultural quality and yield, Solanum lycopersicum cv M82 (WT) and homozygous T3 leeix1 independent CRISPR lines: slnrc4a-1-4 and slnrc4a-1-b5 were grown from seeds in soil (Green Mix; EvenAri, Ashdod, Israel) in a growth chamber, under long-day conditions (16 h:8 h, light:dark) at 24° C. Plants from both independent CRISPR lines were used in all assays. Growth measurement were performed as previously described by Gur et al Plant vegetative weight was determined by weighing only the vegetative tissue (after harvesting the fruits) without the roots. Total fruit yield per plant included both the red and the green fruits. Concentrations of total soluble sugars were measured as BRIX percentage on a digital refractometer with a range of BRIX 0-85%+0.2%, from a random sample of 5 red fruits per plant. Harvest index (HI) was calculated as the ratio between the total yield and total biomass. Reference is now made to FIG. 2a-2e depicting similar agricultural quality and yield of leeix1 mutants as the background M82 line. Agricultural and developmental parameters were measured in M82 and leeix1 plants. FIG. 2a depicts the Harvest index (HI) of plants was calculated as the ratio between the total mass of fruit yield and the total biomass. FIG. 2b depicts the total soluble sugars measured using a refractometer and are expressed as Brix. FIG. 2c depicts the total fruit weight per plant. FIG. 2d depicts average total number of tomato fruits produced per plant. FIG. 2e depicts the analysis of growth and development parameters: height, length of nodes 2 and 3, number of leaves, number of leaves produced in the vegetative stage (before the first flower), and Number of florets. Average ±SEM of at least four independent replicates is shown, N=18. No statistically significant differences were observed among WT (Wild Type) and leeix1 (t-test, welch correction). Reference is now made to FIG. 3a-3d depicting similar agricultural quality and yield of leeix1 mutants as the background M82 line. Agricultural and developmental parameters were measured in M82 and leeix1 plants of lines 1˜4 and lbs. FIG. 3a depicts the Harvest index (HI) of plants which was calculated as the ratio between the total mass of fruit yield and the total biomass. FIG. 3b depicts the total soluble sugars were measured using a refractometer and are expressed as Brix. FIG. 3c depicts the total fruit weight per plant. FIG. 3d depicts the average total number of tomato fruits produced per plant. Average ±SEM of at least three independent replicates is shown, N=8. No statistically significant differences were observed among WT and leeix1 lines except in the case of Brix, where line 1-4 had increased soluble sugars (t-test, Welch's correction, ***p<0.001). It is clear that both leEIX1 mutated plant lines have similar or even better agricultural traits in comparison to the WT M82 background line. A plant line with known and desired agricultural traits that is more resilient due to its increased immune response to pathogenic microorganisms is highly sought after in agrotechnology and plant biotechnology. Such a plant line can help reducing the use of herbicides and pesticides, reduce the crops' losses and prolong crops' shelf life.

### Example 3

The system of the present invention further discloses that leeix1 mutants display lower disease levels compared to the WT plants regarding both necrotrophic fungi and biotrophic fungal pathogens. In order to asses diseases levels B. cinerea isolate BcI16 (necrotrophic) and S. sclerotiorum isolate Sc15 (biotrophic) were cultured on potato dextrose agar (PDA) (Difco Lab) plates and incubated at 22° C. for 5-7 days. B. cinerea spores were harvested in 1 mg ml⁻¹ glucose and 1 mg ml⁻¹ K₂HPO₄ and filtered through cheesecloth. Spore concentration was adjusted to 10⁶ spores ml⁻¹ using a haemocytometer. Leaves 4-6 from 5 to 6-week old tomato plants were excised and immediately placed in humid chambers. Each tomato leaflet was inoculated with two droplets of 10 μL spore suspension.

For S. sclerotiorum, uniform mycelial plugs (5 mm diameter) were taken using a cork-borer from colony margins and placed mycelial side down on the adaxial surface of each leaf. Inoculated leaves were kept in a humid growth chamber at 21° C.

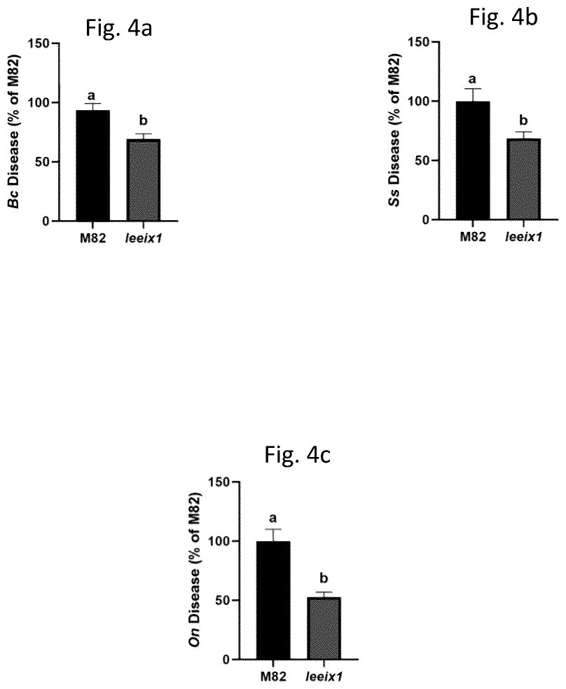

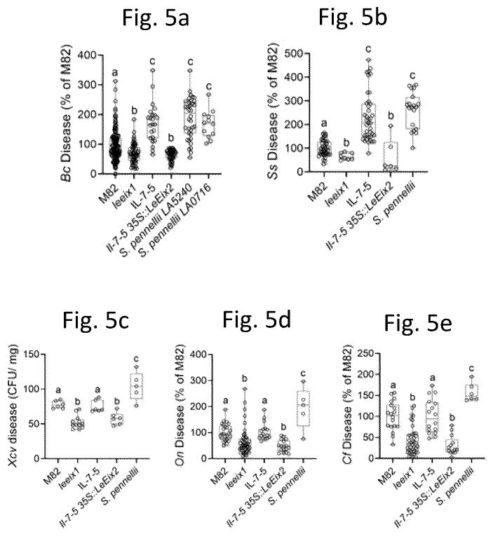

O. neolycopersici (biotrophic) was isolated from young leaves of 4-6 weeks old tomato plants grown in a commercial greenhouse in the winter of 2019. Conidia of the pathogen were collected by rinsing infected leaves with sterile water. For the artificial infection of tomato leaves, the concentrations of conidial suspensions were determined under a light microscope using a hemacytometer. All suspensions were adjusted to 10⁴ ml⁻¹ and sprayed onto 5-6-week old tomato plants at a rate of 5 ml per plant. Suspensions were sprayed within 10-15 min of the initial conidia collection, with a hand-held spray bottle, and plants were left to dry in an open greenhouse for up to 30 min. Inoculated plants were kept in a humid growth chamber at 21° C. In all cases, controls consisted of leaves treated with water/buffer without the inoculation of pathogen. The diameter of the necrotic lesions or % of infected leaf tissue was measured three to ten days post inoculation, as indicated, using the image processing tool ImageJ. Reference is now made to FIG. 4a-4c depicting leeix1 mutants having an improved disease resistance as the lesion area on the leEIX1 mutant's leaves is significantly smaller and more confined, regardless to the fungal inoculation type. Reinforcing the notion that the LeEIX1 mutated plants having an increased immune response towards both necrotrophic and biotrophic pathogens. WT M82 and leeix1 plants were challenged with B. cinerea (Bc) (FIG. 4a) (10⁶ spores/ml), S. sclerotiorum (Ss) (FIG. 4c) (mycelial plugs from 5 days old plates), or O. neolycopersici (On) (FIG. 4c) (10⁴ spores/ml). relative disease area was calculated as the lesion area measured 5 days after inoculation for Bc and Ss, and 10 days after inoculation for On, in each genotype. Bc: Average ±SEM of 6 independent replicates is shown, N=85. Ss: Average ±SEM of 3 independent replicates is shown, N=20. On: Average ±SEM of 3 independent replicates is shown, N=12. Letters represent statistical significance between the WT and leeix1 samples in a one-way analysis of variance with a Bonferroni post-hoc test, p<0.03.

### Example 4

Reference is now made to FIGS. 5a-5e depicting LEEIX1 mutants and LeEIX2 overexpressing plants having improved resistance to various pathogens, such as B. cinerea, S. sclerotiorum, X. euvesicatoria, O. neolycopersici, and C. fulvum. Tomato plants of the indicated genotypes were challenged with B. cinerea (10⁶ conidia/mL) (FIG. 5a), S. sclerotiorum (mycelial plugs from 5 day old plates) (FIG. 5b), X. euvesicatoria (10⁴ CFU/mL) (FIG. 5c), O. neolycopersici (10⁴ conidia/mL) (FIG. 5d), and C. fulvum (10⁶ conidia/mL) (FIG. 5e). Relative disease area was calculated as the lesion area measured 5 days after inoculation in each genotype for B. cinerea and S. sclerotiorum, and similarly, 10 days after inoculation, for O. neolycopersici and C. fulvum. For X. euvesicatoria, pathogen levels were quantified 3 days after inoculation. Boxplots are displayed with inner quartile ranges (box), outer quartile ranges (whiskers), and median (line in box), all points shown. Different letters represent statistically significant differences in a one-way ANOVA with a Tukey post hoc test (FIG. 5a-c, and FIG. 5e), or in two-tailed t-tests (FIG. 5d). In all cases, experiments were repeated 3-6 independent times. (a) N>12, p<0.012 (b) N>6, p<0.0007 (c) N>6, p<0.03 (d) N>7, p<0.05 (e) N>6, p<0.006.

### Example 5

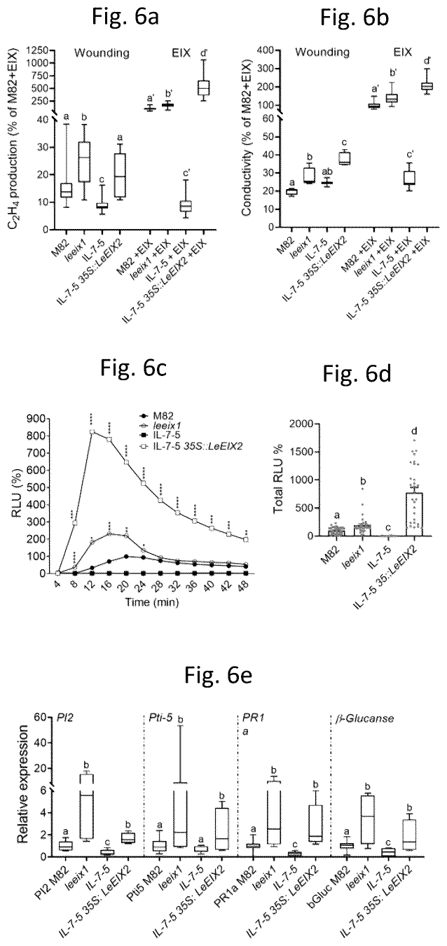

The leeix1 mutants and LeEIX2 of the present invention overexpressing plants have increased defense responses following wounding and to the Trichoderma elicitor Xyn11/EIX, when compared with the WT M82 background line. Reference is now made to FIG. 6a-6e depicting leeix1 mutants and LeEIX2 overexpressing plants having increased defense compared with the WT plants. FIG. 6a depicts a graphical presentation of the ethylene induction after wounding and EIX elicitation in the indicated tomato genotypes, which was measured using gas chromatography. Average ethylene production in M82 was defined as 100%. FIG. 6b depicts a graphical presentation of the measurement of the conductivity of samples derived from the indicated genotypes following wounding or EIX treatment. M82 average conductivity following EIX treatment was defined as 100%. FIG. 6c and FIG. 6d depict graphical presentations of the ROS production in indicated genotypes, which was measured immediately after EIX application every four minutes, using the HRP-luminol method, and expressed as Relative Luminescent Units (RLU). The average peak of ROS production in M82 was defined as 100%. In all cases, experiments were repeated 3 independent times. FIG. 6e depicts a graphical presentation of the measurement, by RT-qPCR, of the gene expression analysis of the defense genes P12, Pti-5, PR1a and beta-glucanase in the indicated genotypes in steady state. M82 expression level was set to 1. (FIG. 6a-b, and FIG. 6e) Boxplots are displayed with inner quartile ranges (box), outer quartile ranges (whiskers), and median (line in box). Different letters represent statistically significant differences among samples, regular letters for wounding and tagged letters for EIX treatment, in Welch's ANOVA with a Dunnett post hoc test (FIG. 6a) N>12, p<0.018. (FIG. 6b) N>8, p<0.01, or in a Welch's t-test or a Mann-Whitney U test comparing each gene among genotypes, N>4, p<0.048 (FIG. 6e). (FIG. 6c) Kinetics from 3 independent experiments are presented, N=26. Asterisks indicate significant upregulation from ROS production in M82 in multiple t-tests with Holm-Sidak correction, *p<0.05, **p<0.01, ***p<0.001, ****p<0.0001. (FIG. 6d) Graph indicates average ±SEM with all points shown. Different letters represent statistically significant differences in Kruskal Wallis ANOVA with Dunn's post hoc test, N=26, p<0.0001.

Examples 4 and 5 together demonstrate that leeix1 mutant is disease resistant on its own, without the involvement of trichoderma.

### Example 6

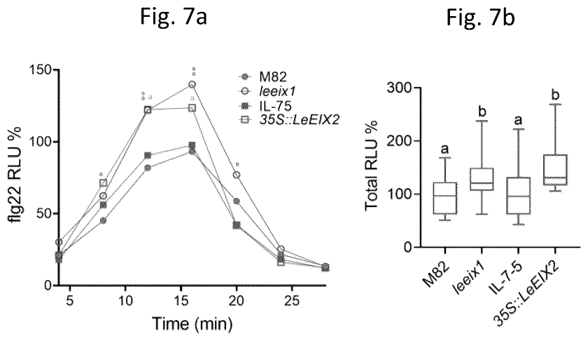

As disclosed above, the leeix1 mutants and LeEIX2 overexpressing plants of the present invention have increased defense responses to the bacterial elicitor flg22, generating significantly higher levels of ethylene, conductivity and reactive oxygen species (ROS) in response to EIX treatment. Reference is now made to FIG. 7a and FIG. 7b depicting leeix1 mutants and LeEIX2 overexpressing plants have increased defense responses to the bacterial elicitor flg22. ROS production in indicated genotypes was measured immediately after flg22 application every four minutes, using the HRP-luminol method. M82 average ROS peak production was defined as 100%. The experiment was repeated 4 independent times. (FIG. 7a) Kinetics from 4 independent experiments are presented, N=48. Circles and squares indicate significance from M82 ROS production in multiple t-tests with Holm-Sidak correction, oi. p<0.05, oo/oo p<0.01, circles for leeix1 and squares for 35S::LeEIX2. (FIG. 7b) Boxplots indicate minimum to maximum values of total ROS production in Relative Luminescent Units (RLU), box indicates inner quartile ranges, whiskers indicate outer quartile ranges, and line in box indicates median. Different letters represent statistically significant differences in a one-way ANOVA with a Tukey post hoc test, N=48, p<0.023.

### Example 7

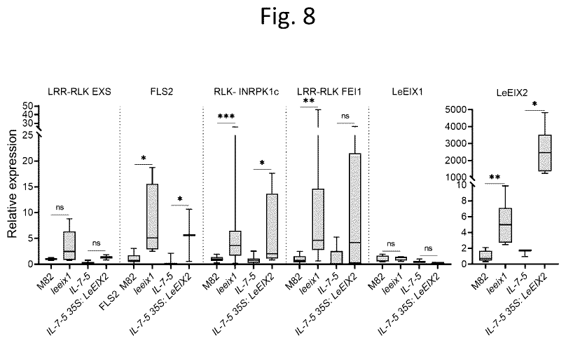

The present invention also proved that PRRs are induced upon leeix1 knockout and LeEIX2 overexpression. Reference is now made to FIG. 8 depicting the gene expression analysis of PRR genes in the indicated genotypes, which was measured by RT-qPCR. Relative expression was calculated using the geometric mean between the gene copy number obtained for two reference genes: RPL8 (Solyc10g006580) and CYP (SolycOlg111170), and normalized to the background line: M82 for leeix1, and IL-7-5 for IL-7-5 35S::LeEIX2. Boxplots indicate minimum to maximum values of gene expression, with box indicating inner quartile ranges, whiskers indicating outer quartile ranges, and line in box indicating median. Asterisks represent statistical significance in t-test with Welch's correction comparing the expression of each gene in each genotype with the expression of that gene in the background line, in three independent experiments (*p-value<0.05; * *p-value<0.01; * * *p-value<0.001).

### Example 8

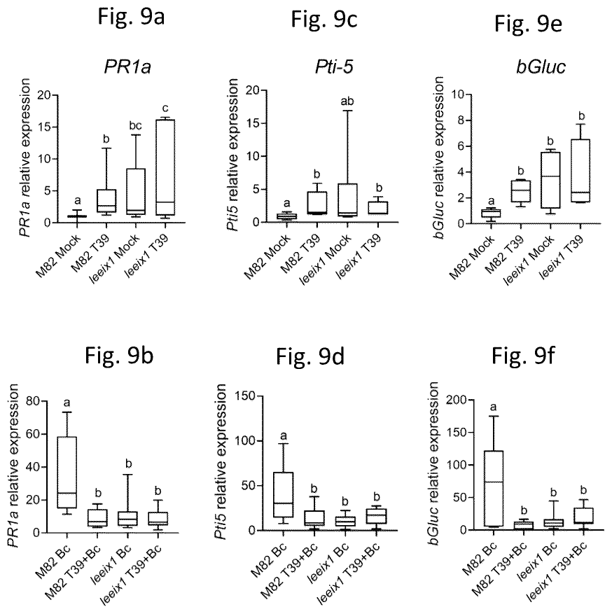

In the present invention, the defense gene expression is increased in leeix1 mutants in steady state, and decreased in leeix1 mutants during pathogenesis. Reference is now made to FIGS. 9a-f depicting defense gene expression is increased in leeix1 mutants in steady state, and decreased in leeix1 mutants during pathogenesis. The Gene expression analysis of defense genes in M82 and leeix1 plants was measured by RT-qPCR, in mock and T harzianum T39 treated samples, 3 days after treatment with 10⁶ conidia/mL (FIGS. 9a,c, e), and 24 h after subsequent B. cinerea (Bc) infection (FIGS. 9b,d, f). Relative expression was calculated using the mean between the gene copy number obtained for two reference genes: RPL8 (Solyc10g006580) and CYP (SolycO1g111170), and normalized to M82 Mock. Boxplots indicate minimum to maximum values of gene expression, with box indicating inner quartile ranges, whiskers indicating outer quartile ranges, and line in box indicating median. Different letters represent statistically significant differences from M82 Mock (FIGS. 9a,c, e) or M82 infected with Bc (FIGS. 9b,d, f) in a t-test with Welch's correction comparing each gene, in three independent experiments. (a, b) PRla. N=6, p<0.045. (FIGS. 9c,d) Pti-5. N=6, p<0.041. (e, f) bGluc. N=3, p<0.046.

### Example 9

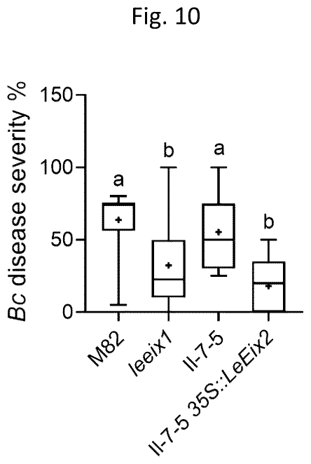

Furthermore, the leeix1 mutants and LeEIX2 overexpressing plants of the present invention retain increased resistance to B. cinerea in sterile conditions. Reference is now made to FIG. 10 depicting leeix1 mutants and LeEIX2 overexpressing plants retain increased resistance to B. cinerea in sterile conditions. Tomato plants of the indicated genotypes were grown in sterile conditions and challenged with B. cinerea (10⁶ conidia/mL). Relative disease area was calculated as the lesion area measured 5 days after inoculation. Boxplots are displayed with inner quartile ranges (box), outer quartile ranges (whiskers), median (line in box), and mean (“+”). Different letters represent statistically significant differences in a one-way ANOVA with a Tukey post hoc test. Experiment was conducted 6 independent times, N=18, p<0.02.

TABLE 1
Primer pairs used for the present invention and Cas9 gRNA primers

| Locus | Name | Forward | Reverse |
|---|---|---|---|
| Solyc01g106620 | PR1a | CTGGTGCTGTGAAGATGTG | TGACCCTAGCACAACCAAG |
| | | G | A |
| Solyc03g020050 | Proteinase | CGACGTGTTGCACTGGTT | TGCCAATCCAGAAGATGGA |
| | inhibitor 2 | AC | C |
| Solyc01g060020 | beta- | TCGAACAGGAGGAGGATC | TCCAGGCTTTCTCGGACTA |
| | 1,3 | TG | C |
| | glucanase | | |
| Solyc02g077370 | Pti 5 | GACATGGTGCGAGAGTATG | CTGAAACAGAGGCGTTCAC |
| | | G | T |
| Solyc10g006580 | RPL8 | TGGAGGGCGTACTGAGAA | TCATAGCAACACCACGAAC |
| | | AC | C |
| Solyc07g008620 | LeEix1gRNA | taggtctccCTCAAGCAGAG | taggtctccTGAGTTGGA |
| | | AAttttagagctagaaat | GTtgcaccagccgggaa |
| L5AD5 | tRNA-gRNA | CGGGTCTCAGGCAGGATGG | TAGGTCTCCAAACGGATGA |
| | Cas9 plasmid | GCAGTCTGGGCAACAAAGC | GCGACAGCAAACAAAAAAA |
| | | ACCAGTGG | AAAGCACCGACTCG |
| S5AD5 | tRNA-gRNA | CGGGTCTCAGGCAGGATGG | TAGGTCTCCAAACGGATG |
| | Cas9 plasmid | GCAGTCTGGGCA | AGCGACAGCAAAC |

The invention is not intended to be limited to the embodiment illustrated and described above, but it can be modified and varied within the scope and spirit of the invention as defined by the following claims.

1-58. (canceled)

59. A mutated plant line characterized by an increased defense response compared to a non-mutated plant of the same population, wherein said mutated plant harbors an impaired LeEIX1 (Lycopersicon esculentum ethylene inducing xylanase receptor 1) gene.

60. A mutated plant line characterized by reduction in disease levels compared to a non-mutated plant of the same population upon pathogen exposure, wherein said mutated plant harbors an impaired LeEIX1 (Lycopersicon esculentum ethylene inducing xylanase receptor 1) gene.

61. The mutated plant line according to claim 59, wherein said mutated plant line is homozygous to the impaired LeEIX1 gene, and/or said mutated plant line expresses a non-functional, truncated LeEIX1 protein.

62. The mutated plant line according to claim 59, wherein said plant is a member of the Solanaceae family.

63. The mutated plant line according to claim 59, wherein said plant is selected from the group consisting of Solanum lycopersicum, Nicotiana tabacum, Solanum tuberosum, Solanum melongena, Capsicum annum, Petunia hybrida, a LeEIX1 gene expressing plant, a plant expressing a LeEIX1 functional ortholog, and any combination thereof.

64. The mutated plant line according to claim 59, wherein said increased defense response is further increased upon interaction with either a pathogen or a bio control agent (BCA).

65. The mutated plant line according to claim 60, wherein the reduction in disease levels is further increased by a bio control agent (BCA).

66. The mutated plant line according to claim 60, wherein said pathogen is selected from the group consisting of viruses, bacteria, fungi, oomycetes, pests and any combination thereof.

67. The mutated plant line according to claim 60, wherein said pathogen is selected from the group consisting of: Botrytis, Sclerotinia, Sclerotium rolfsii, Alternaria, Pythium, Phytopthora, Fusarium, Oidium, Lasodiplodia, Penicillium, Aspergillus, Talaromyces, Macrophomina, Verticillium, Cladosporium, Clavibacter, Pseudomonas, Xanthomonas, Xyllela, Erwinia, Tobamoviruses, Tomato yellow leaf curl virus, Gemini viruses, Flies, Mites, Leafhoppers, Leafminers.

68. The mutated plant line according to claim 64, wherein said agent is selected from the group consisting of:
insects;
microorganisms such as fungi, bacteria, oomycets, viruses; and
chemicals such as nucleic acids, hormones, hormone analogs, natural products, organic compounds.

69. The mutated plant line according to claim 64, wherein said agent is Trichoderma harzianum.

70. The mutated plant line according to claim 64, wherein said agent is T39.

71. The mutated plant line according to claim 59, wherein said mutated plant line is mutated by means of a gene editing system.

72. The mutated plant line according to claim 71, wherein said gene editing system is selected from the group consisting of: gene editing agent, meganucleases, zinc finger nucleases, TALEN, Crispr/Cas and any combination thereof.

73. The mutated plant line according to claim 71, wherein said gene editing system is crispr/cas 9 system.

74. A method for increasing a plant defense response, said method comprising the following steps:
designing at least one nucleic acid sequence configured to targeting and impairing the LeEIX1 gene; and
applying a gene editing process utilizing said at least one nucleic acid sequence.

75. The method according to claim 74, said method further comprising the step of obtaining at least one mutant plant line, harboring an impaired LeEIX1 gene.

76. The method according to claim 74, said method further comprising the following steps:
obtaining at least one mutant plant line, harboring an impaired LeEIX1 gene;
growing said plant; and
inoculating said plant with at least one bacterial biological control agent.

77. The method according to claim 74, wherein said nucleic acid sequence is selected from the group consisting of DNA, RNA, tRNA, gRNA, cDNA, hybrid nucleic acid, and any combination thereof.

78. The method according to claim 74, wherein said mutant plant line possesses at least one increased defense response.
